(19) United States
(12) Patent Application Publication
(10) Pub. No.: US 2011/0218498 A1
(43) Pub. Date: Sep. 8, 2011

(54) CLEATED ANCHORING SYSTEM

(75) Inventors: Steven F. Bierman, Del Mar, CA (US); Richard A. Pluth, San Diego, CA (US)

(73) Assignee: VENETEC INTERNATIONAL, INC., Covington, GA (US)

(21) Appl. No.: 13/057,731

(22) PCT Filed: Aug. 6, 2008
(86) PCT No.: PCT/US08/72370

§ 371 Date: May 24, 2011

(51) Int. Cl. A61M 25/02

(52) U.S. Cl. 604/180; 604/174

(57) ABSTRACT

A securement device (2) for a medical article (50) includes an anchor pad (6), a retainer (9), a cleat (16), and a filament (25). The retainer (9) can be supported by the anchor pad (6) and have an upper member (30) and a lower member (10). The securement device (2) includes one or more filaments (25) coupled to the retainer (9). The filament (25) can further tie about a portion of a medical article (50) and secure about the cleat (16). Below the upper member, the securement device (2) can include a biasing member that flexes relative to the retainer (9).

### BACKGROUND OF THE INVENTION

1. Field of the Invention

The invention relates to securement devices; and, more particularly, to anchoring systems for anchoring or securing medical articles to the body of a patient.

2. Description of the Related Art

It is often desirable to provide a securement device capable of securing a wide variety of catheter shapes and sizes, as well as other medical articles. Operating conditions require that the devices be secured relatively firmly to avoid complications.

### SUMMARY OF THE INVENTION

There is a need for a securement device for percutaneous sheath introducers and other medical devices that uses flexible strands for securement means thus allowing versatility and flexibility and does not require careful and expensive manufacturing of the medical articles to be secured. Such a device should easily accommodate catheters of varying shapes and sizes. Further, it may be advantageous for the securement device to comprise a mechanism to retain tension in the strands.

An aspect of the invention involves a securement device for a medical article. The device includes an anchor pad having a bottom surface and a top surface. At least a portion of the bottom surface is covered by adhesive. The device further includes a base and a bed of unitary or separate construction. The base is supported by the anchor pad. The device further includes at least one biasing member. At least a portion of the biasing member is disposed below the bed and has a generally fixed base and a distal portion that flexes relative to the base. The device further includes at least one cleat and at least one filament coupled to the biasing member that has a free end. The free end is configured to be tied about a portion of the medical article and secured relative to the cleat.

Another aspect involves a retainer for securing a medical article. The retainer includes a support that has an upper member supported at least in part by a lower member. The lower member is harder than the upper member. The retainer further includes at least one aperture and a cleat extending from the lower member and has at least one slot. The retainer further includes a filament coupled to the lower member through the at least one aperture and having a free end. At least a portion of the filament is configured to wrap about the cleat and engage with the slot.

Another aspect involves a securement device for a medical article. The device includes a retainer that has at least two materials with at least one of the two materials being softer than the other one of the materials. The device further includes at least one cleat having a slot and at least one filament permanently coupled to the retainer and has a free end. The free end is configured to be wrapped about a portion of the medical article and secured through the slot of the cleat.

These and other aspects of the present invention will become readily apparent to those skilled in the art from the following detailed description of the preferred embodiments, which refers to the attached figures. The invention is not limited, however, to the particular embodiments that are disclosed.

### BRIEF DESCRIPTION OF THE DRAWINGS

These and other features, aspects, and advantages of the invention disclosed herein are described below with reference to the drawings of preferred embodiments, which are intended to illustrate and not to limit the invention. Additionally, from figure to figure, the same reference numerals have been used to designate the same components of an illustrated embodiment. The following is a brief description of each of the drawings.

FIG. 1 illustrates a perspective view of a securement device in accordance with a preferred embodiment of the present invention;

FIG. 2 is an exploded, perspective view of the securement device from FIG. 1 showing base and bed portions both aligned with an anchor pad;

FIG. 3 is a partially exploded, perspective view of the securement device from FIG. 2 with the base portion disposed on the anchor pad;

FIG. 4 illustrates a perspective view of the base portion of the securement device from FIG. 2;

FIG. 5 illustrates a top plan view of the base portion from FIG. 4;

FIG. 6 illustrates a side view of the base portion from FIG. 5;

FIG. 7 illustrates a perspective, bottom view of the base portion from FIG. 4;

FIG. 8 illustrates a perspective view of the bed portion of the securement device from FIG. 2;

FIG. 9 illustrates a top plan view of the bed portion from FIG. 8;

FIG. 10 illustrates a side view of the bed portion from FIG. 9;

FIG. 11 illustrates a perspective, bottom view of the bed portion from FIG. 8;

FIG. 12 illustrates a top plan view of the retainer of the securement device of FIG. 1;

FIG. 13 illustrates a cross-sectional side view of the retainer along line 13-13 of FIG. 12;

FIG. 13A illustrates an enlarged view of a portion of the retainer from FIG. 13;

FIG. 14 illustrates a cross-sectional side view of the retainer along line 14-14 of FIG. 12;

FIG. 15 illustrates a medical article in position to be received by the securement device of FIG. 1;

FIG. 16 illustrates the medical article received by the securement device of FIG. 1 with a single end of the strand extending through the medical article and securement device;

FIG. 17 illustrates a perspective view of a securement device according to another preferred embodiment of the present invention;

FIG. 18 illustrates a perspective view of a bed portion of the securement device of FIG. 17;

FIG. 19 illustrates a perspective view of a securement device according to another preferred embodiment of the present invention;

FIG. 20 illustrates a perspective view of the bed portion of the securement device of FIG. 19;

FIG. 21 illustrates another medical article in position to be received by the securement device of FIG. 1;

FIG. 22 illustrates the medical article of FIG. 15 secured by the securement device of FIG. 1;

FIG. 23 illustrates the medical article of FIG. 15 further secured by the securement device of FIG. 1;

FIG. 24 illustrates the medical article of FIG. 15 secured by the securement device of FIG. 1 in a manner that is different from what is illustrated in FIG. 23;

FIG. 25 illustrates one tying arrangement of a single end of the strand about the cleat; and

FIG. 26 illustrates a perspective view of a securement device according to another preferred embodiment of the present invention.

### DETAILED DESCRIPTION OF THE PREFERRED EMBODIMENTS

The following description and examples illustrate preferred embodiments of the present anchoring system in detail which is disclosed in the context of use with an exemplary medical device, such as a junction. The principles of the present invention, however, are not limited to medical devices. It will be understood by those of skill in the art in view of the present disclosure that the anchoring system described can be used with other types of medical articles, including, but not limited to: other junctions, catheters, fluid delivery tubes, electrical wires, and other medical devices or their components. One skilled in the art may also find additional applications for the devices and systems disclosed herein. Thus, the illustrations and descriptions of the anchoring system in connection with the medical devices are merely exemplary of some possible applications of the anchoring system.

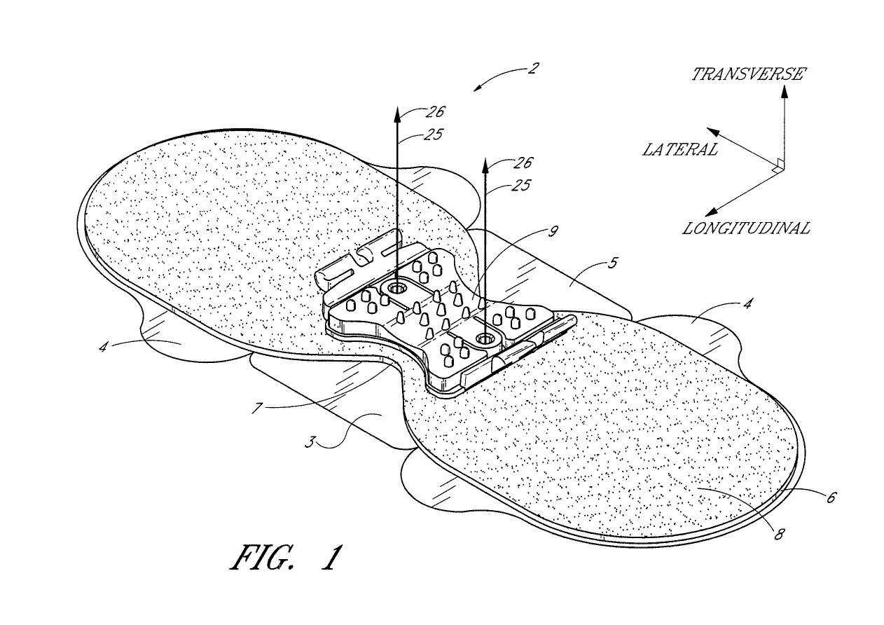

To assist in the description of these components of the anchoring system, the following coordinate terms are used. A “longitudinal axis” is generally parallel to the groove in the securement device 2 and the major axis of the medical article (further described below). A “lateral axis” is normal to the longitudinal axis and is generally parallel to the plane of an anchor pad 6, as seen in FIG. 1. A “transverse axis” extends normal to both the longitudinal and lateral axes. In addition, as used herein, “the longitudinal direction” refers to a direction substantially parallel to the longitudinal axis; “the lateral direction” refers to a direction substantially parallel to the lateral axis; and “the transverse direction” refers to a direction substantially parallel to the transverse axis. The terms “upper,” “lower,” “top,” “bottom,” and the like, which also are used to describe the present anchoring system, are used in reference to the illustrated orientation of the embodiment. A detailed description of a preferred embodiment of the anchoring system, and its associated method of use, now follows.

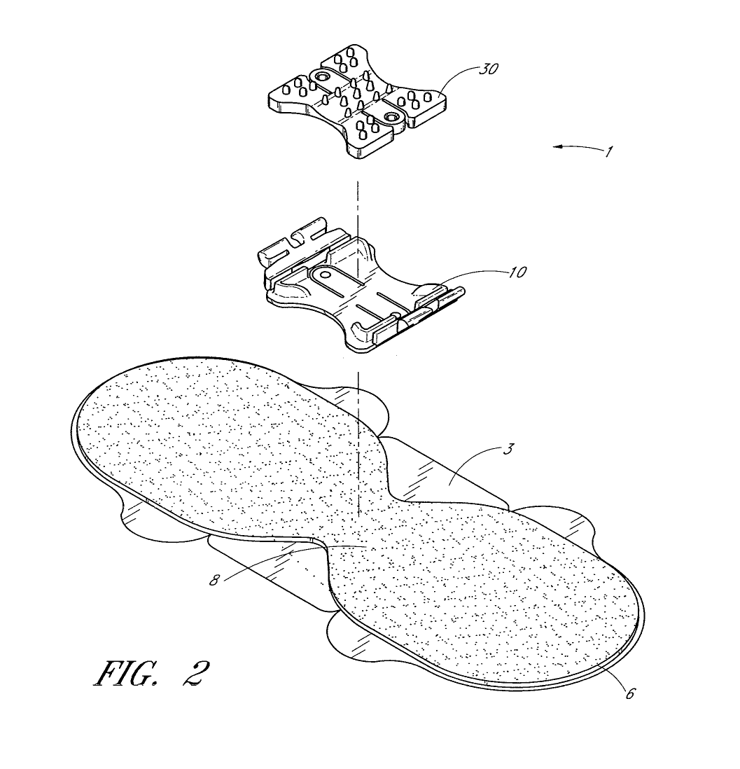

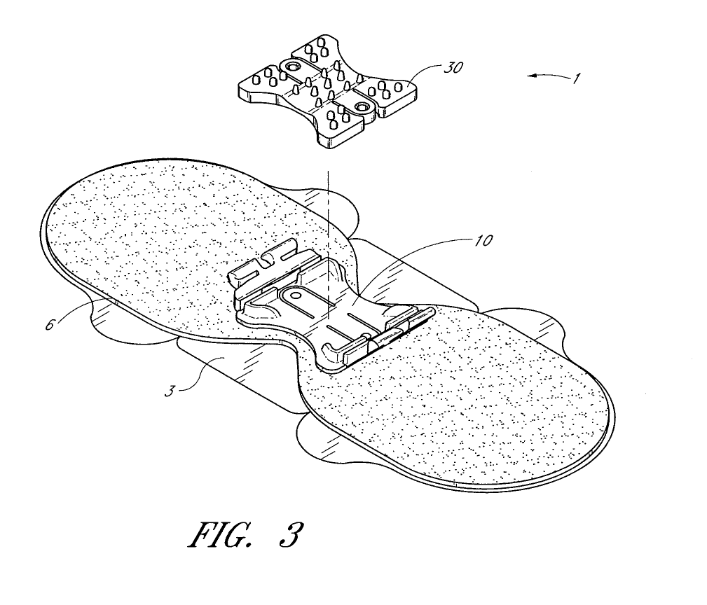

As shown in FIG. 1, the described embodiment comprises a securement device 2 in two main components: the anchor pad 6 and the retainer 9. The retainer 9 comprises several sub-components, including a base 10, a bed 30, and one or more retaining members, as depicted in FIGS. 2, 3. These sub-components may be of unitary or separate construction. For example, the base 10 and bed 30 may be unitary but separate from the retaining members. Alternatively, they may all be of unitary construction. The securement device 2 preferably includes at least one flexible strand or filament 25. While the term flexible may be used to describe the strand 25, only a portion(s) of the strand 25 need be flexible. The flexible portion(s) of the strand 25 allows the strand 25 to be tied together or around a cleat 16 so as to secure the medical article 12 to the securement device 2.

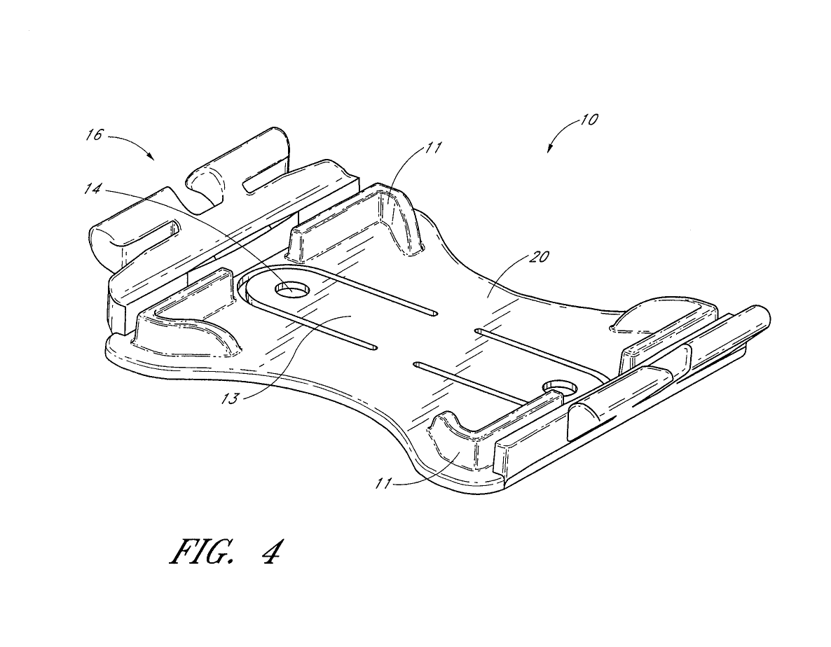

The securement device 2 may include one or more strands 25. The strand 25 interfaces with the retainer 9. Each strand 25 may form one or two ends that are secured to one or more cleats 16. The one or more strands 25 may form a unitary structure with the retainer 9 or be separately manufactured and assembled with the retainer 9. In the illustrated embodiments, the strand 25 is separately manufactured and assembled with the retainer 9 via holes 14, 34 (depicted e.g. in FIGS. 4, 9). The strand 25 in the embodiment illustrated in FIG. 1 forms two ends 26. At least one of the ends is secured to the one or more cleats 16 (depicted e.g. in FIG. 4) at least when the medical article is secured to the device 2.

In the illustrated embodiment, the holes 14, 34 together form a passageway through the base 10 and bed 30 of the retainer 9. The strand 25 extends through the holes 14, 34, wraps about the medical article 50, and secures to the cleat 16. The strand 25 need not secure to a clip, cleat 16, a cinch, or any other suitable connector and may instead secure to itself by means of a knot, hook, or other mechanism.

The anchoring system 1, including the securement device 2 and the medical article 50, can form a component of a medical device system that also includes one or more medical articles (e.g., a junction). The retainer 9 is mounted upon the anchor pad 6 and the anchor pad is secured to the skin of the patient, generally by an adhesive disposed upon the bottom surface of the pad. The retainer 9 receives the medical article 50 and secures it in position, as shown in FIG. 15. In a preferred embodiment the releasable engagement of the medical article 50 is achieved, at least in part, by cooperation of the bed 30 and retaining members. Because generally the retaining members reversibly secure the medical article 50, it is possible for the medical article to be removed from the anchoring system 1 for any purpose, such as to replace the anchoring system or to facilitate moving the patient. This removal of the medical article 50 from the anchoring system 1 can be accomplished without removing the anchoring system from the patient if desired.

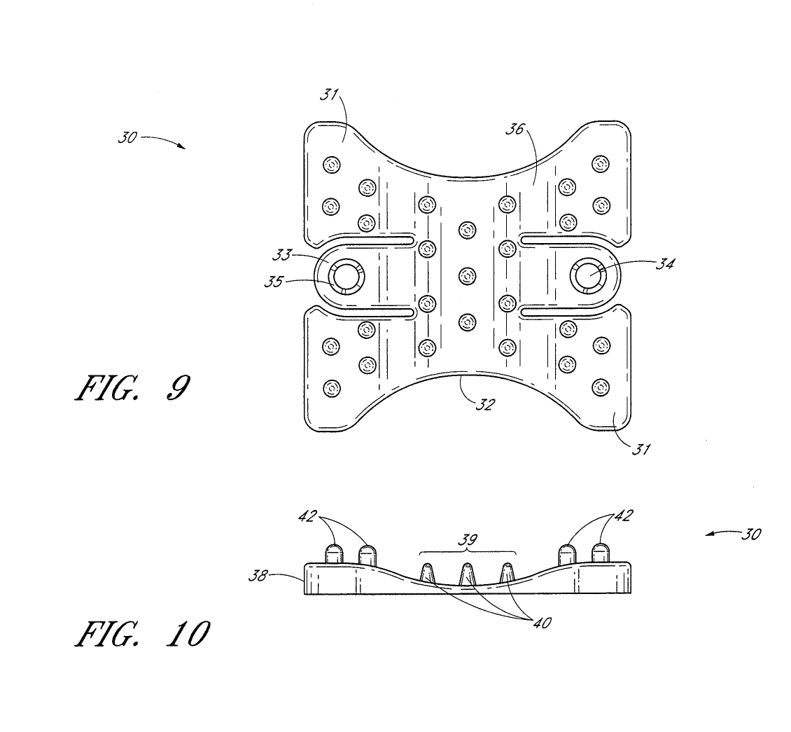

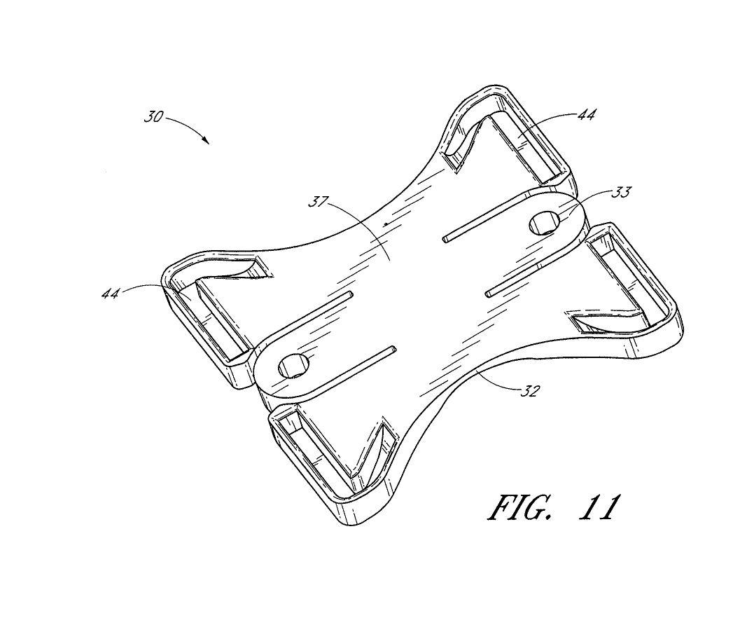

The medical article 50 is held in position through combinations of lateral, longitudinal, and transverse pressures along the secured portion of the medical article 50 within a portion of the retainer 9. As illustrated in FIGS. 8, 10, the retainer 9 preferably comprises a groove 39 disposed in the bed 30. Pressure is provided to the sides of the retained medical article 50 by the walls of the groove 39 and the retaining members described below. When the medical article 50 is secured, these forces inhibit the article from moving substantially in either the lateral or transverse directions. Longitudinal motion of the medical article 50 can be inhibited at least partially by friction of the retaining members against the article.

Furthermore, the embodiment described provides a universal feature such that the anchoring system 1 can be used to receive and secure a variety of sizes and shapes of medical articles. Because the securing forces can be provided in part by the flexible strand 25, a variety of medical articles 50 of varying transverse heights and shapes may be accommodated. Furthermore, the strand 25 may be used to hold multiple medical articles 50, or for example bundles thereof.

The anchoring system 1 also desirably releasably engages the medical article 50. This allows the anchoring system 1 to be disengaged from the medical article 50 without removing the medical article. For instance, the healthcare provider may wish to remove the anchoring system 1 in order to change the anchor and/or anchor pad, or clean the patient without removing the medical article 50 from the patient. In situations where the medical article 50 is in position in the patient for an extended period of time, it may be advantageous to periodically change the anchor and/or anchor pad while still maintaining the best positional securement of the medical article 50. For these purposes, it is desirable that the disengagement of the medical article 50 from the anchoring system 1 can be accomplished without removing the medical article 50 from the patient.

With reference now to FIG. 1, the anchoring system 1 includes anchor pad 6, a retainer 9, and a strand 25. The anchor pad 6 secures the retainer 9 to a patient's skin. The anchor pad 6 has a lower adhesive surface which adheres to the skin of a patient and an optionally roughened upper surface 8 which supports a retainer 9. The retainer 9 is configured to accept and retain a section of a medical article 50 within the anchoring system 1. As shown in FIGS. 2-3, the retainer 9 comprises a base 10 and a bed 30. The bed 30 may be, but need not be, releasably secured to the base 10.

The anchor pad 6 desirably comprises a laminate structure with an upper foam layer (e.g., closed-cell polyethylene foam) and a lower adhesive layer. The lower adhesive layer constitutes the lower surface of the anchor pad 6. The lower surface desirably is a medical-grade adhesive and can be either diaphoretic or nondiaphoretic, depending upon the particular application. Such foam with an adhesive layer is available commercially from Avery Dennison of Painsville, Ohio. Although not illustrated, it will be understood that the anchor pad 6 can include suture holes in addition to the adhesive layer to further secure the anchor pad 6 to the patient's skin.

In an alternative embodiment, a hydrocolloid adhesive may advantageously be used upon the anchor pad 6 for attaching the anchor pad to the skin of the patient. The hydrocolloid adhesive has less of a tendency to excoriate the skin of a patient when removed. This may be particularly important for patients whose skin is more sensitive or fragile, such as those with a collagen deficiency, common to dialysis patients.

As shown, a surface of the upper foam layer constitutes an upper surface 8 of the anchor pad 6. The upper surface 8 can be roughened by corona-treating the foam with a low electric charge or by chemical treatment. The roughened or porous upper surface 8 can improve the quality of the adhesive joint (which is described below) between the base 10 and the anchor pad 6. In the alternative, the flexible anchor pad 6 can comprise a medical-grade adhesive lower layer, an inner foam layer and an upper paper or other woven or nonwoven cloth layer.

A removable paper or plastic release liner 3 desirably covers the adhesive lower surface before use. The liner 3 preferably resists tearing and desirably is divided into a plurality of pieces to ease attachment of the pad to a patient's skin. In the illustrated embodiment, the liner 3 is split into three sections, a middle portion 5 and two side portions including grip portions 4.

The liner 3 length, as measured in the lateral direction, extends beyond the lines 4 and can be folded over, or back onto the liner 3. When folded over, the liner 3 can define a pull tab to facilitate removal of the liner 3 from the adhesive lower surface. In one embodiment, best shown in FIG. 1, the liner 3 extends longitudinally beyond the anchor pad 6 to provide grip sections 4. A medical attendant can use the grip sections 4 by grasping and pulling on them so that the liner 3 is separated from the lower surface of the anchor pad 6. The grip sections 4 can overcome any requirement that the medical attendant pick at a corner edge or other segment of the liner 3 in order to separate the liner 3 from the adhesive layer. The grip sections 4 of course can be designed in a variety of configurations. For example, the grip sections 4 can be located along any portion of the liner 3 in order to ease the application of the anchor pad 6 onto the patient's skin at a specific site. For example, an area of a patient's skin with an abrupt bend, such as at a joint, can require that the grip sections 4 be aligned toward one of the lateral ends of the anchor pad 6. Further, it will be clear from this disclosure that one or more peel lines (not shown) where the liner 3 is folded over can also be used as a grip section 4.

The liner 3 can further be separated into separate sections. As shown for example in FIG. 1, the liner 3 can be separated into 3 sections: a middle portion beneath the retainer 9, and two side portions beneath the periphery of the anchor pad 6. In operation, a medical attendant can first remove the center portion of the liner 3 and affix that portion to the patient. The medical attendant can then retain the medical article 50 to the patient using a strand 25 wrapped about the medical article and secured to at least one cleat 16 (or in another manner described herein). The medical attendant can then peel off the side portions of the liner 3 and apply the corresponding portions of the anchor pad 6 to the patient, fully anchoring the securement device 2 to the patient.

As best seen in FIG. 1, the anchor pad 6 also desirably includes a pair of opposing concave sections 7 that narrows the center of the anchor pad 6 proximate to the retainer 9. As a result, the lateral sides of the anchor pad 6 have more contact area which provides greater stability and adhesion to a patient's skin. As shown, the anchor pad 6 is sized and shaped in the center to approximately match the retainer 9. The anchor pad 6 can thus minimize the area of skin covered by the anchor pad 6 proximal to the retainer 9 and accordingly the medical article 50. Leaving the skin unoccluded can constitute a substantial goal in some applications, such as for example when an IV catheter is to enter the patient proximal to the anchor pad 6. Thus, providing the majority of the adhesive surface far from the retainer 9 advantageously leaves the skin substantially uncovered proximal to the retainer.

The total size of the anchor pad 6 can be chosen taking into account the adhesive used, the medical article 50 to be retained, the expected stresses on the medical article, as well as other considerations. Generally, the force necessary to remove the anchor pad 6 from the skin of the patient will be positively related to both the strength of the adhesive used and the total area of the anchor pad 6. Thus, for example, generally a smaller anchor pad 6 may be used with a stronger adhesive. The nature of the medical article 50 to be retained and the expected stresses thereon can be used to determine how strong a connection between the anchor pad 6 and skin is necessary for the safety of the patient.

Although in preferred embodiments adhesive is used, other attachment elements are available. In accordance with the invention, the anchor pad 6 and/or the retainer 9 (with or without the anchor pad) can be attached to the patient by means such as sutures, elastic bands, Velcro® bands, and other elements.

With reference now to FIGS. 2-3, the retainer 9 comprises a base 10 and a bed 30. As depicted, the base 10 and bed 30 can come together to form the retainer 9. In other embodiments the base 10 and the bed 30 can also be formed integrally. Separating the base 10 and the bed 30 into two pieces may be preferable where the bed 30 must be changed, replaced, or removed relatively frequently. Further, separating the base 10 and bed 30 allows for modular designs involving different bases and beds. Similarly, in some embodiments the base 10 may be formed integrally with the anchor pad 6, or be more rigidly attached. In other embodiments the bed 30 may be movably attached to the base 10, such as being rotatable about a post in the base.

The base 10 and bed 30 may further comprise a variety of materials. As discussed below, in some embodiments the bed 10 and base 30 can comprise elastic materials such as plastic, rubber, silicone, thermoplastic elastomers, and the like. Further, for medical applications the bed 30 and base 10 desirably comprise non-toxic materials. The base 10 can further comprise a more rigid material than the bed 30. Additionally, the bed 30 can comprise a high-friction material.

Further, the base 10 of the retainer 9, as illustrated, is attached to the upper surface 8 of the anchor pad 6. The base 10 desirably is secured to the upper surface 8 by a solvent bond adhesive, such as cyanoacrylate or other bonding material. Such adhesives available commercially are Part No. 4693 from the Minnesota Mining and Manufacturing Company (3M), Loctite®, cyanoacrylate, and the like.

In the embodiment illustrated in FIGS. 2-3, the strand 25 does not include a protuberance or knot positioned between its ends 26. However, the strand 25 may have one or more knots to limit movement of the strand 25 in at least one direction relative to retainer 9. At least a portion of the knot desirably has a diameter which is larger than the diameter of the corresponding hole 14, 34. It is contemplated, however, that the strand 25 can be configured to allow a healthcare provider to form one or more knots in the strand 25.

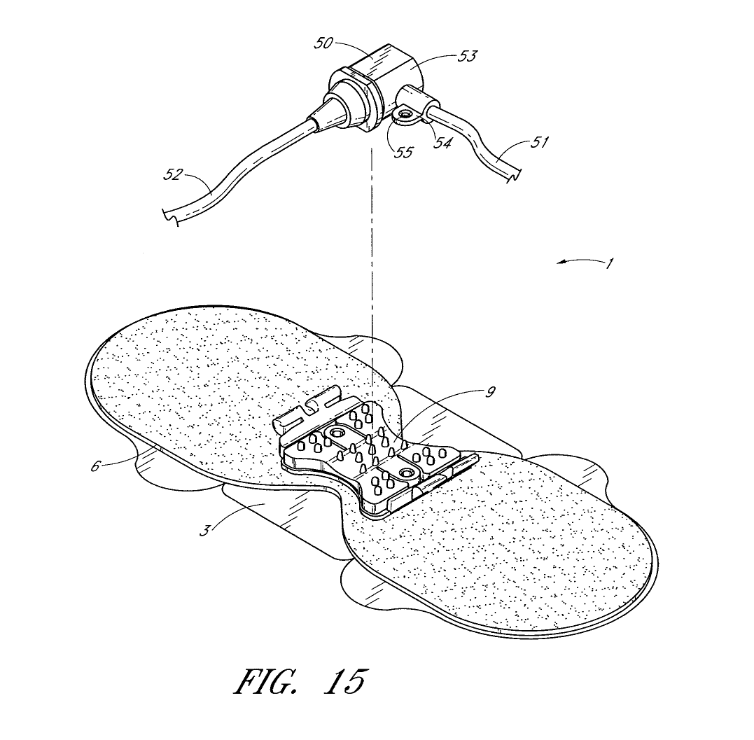

As can be seen in FIG. 13, in the illustrated embodiment of the securement device 2, the portion of the strand 25 extending between the holes 14, 34 is positioned underneath at least a portion of the base 10 and above at least a portion of the anchor pad 6. In such an embodiment, movement of the strand 25 relative to the retainer 9 may be inhibited, such as by friction between the adjacent layers. However, the medical article 50 may be secured even if movement of the strand 25 is permitted by, for example, wrapping the strand 25 about one or both cleats 16.

In the illustrated embodiment, a continuous strand 25 is employed having two exposed ends 26 for securement about the medical article 50. Of course, a single strand 25 may be used with a single end 26 or two separate strands 25 may be used with each forming a single end 26 for securement about the medical article 50. In certain embodiments that have a single strand 25 or multiple separate strands 25, each strand 25 may include a knot or other protuberance. For example, a knot may be positioned on the strand 25 so as to inhibit the strand 25 from sliding through the hole(s) 14, 34. In such an embodiment, the strand 25 need not be continuous between the holes 14, 34 unlike what is illustrated in FIG. 13. The knot may be larger in size than the corresponding hole 14, 34 such that the knot can not be easily pulled through the corresponding hole 14, 34. In other embodiments, the knot may be smaller than the hole 14, 34, but a side of the knot's bulbous outer periphery may catch against a side of the hole(s) 14, 34 to inhibit relative movement in at least one direction.

With reference to FIGS. 4-7, the base 10 comprises a generally flat rectangular form. As will be clear from this disclosure to one skilled in the art, the base 10 may generally resemble other shapes, such as triangles, circles, ovals, other polygons, and other geometric figures. Further, the illustrated embodiment of the base 10 is generally flat along a bottom surface 21. The bottom surface 21 generally should match the anchor pad 6 and the patient. In some embodiments the anchor pad 6 may be flexible, so as to accommodate diversely shaped surfaces. Generally, the base 10 will not be as flexible, but may still be sufficiently flexible for the bottom surface 21 to substantially match the relevant surface of the patient and the anchor pad 6. When a flexible base 10 is desirable, the base 10 may comprise a smaller height or a more flexible material, or either of these adjustments in a bendable portion of the base.

In some embodiments, the bottom surface 21 of the base 10 may comprise a curve or other shape substantially matching a region of the patient to which it is to be attached. The degree to which the base 10 must be custom-fitted can depend on the size and flexibility of the base, as well as the radii of curvature of the relevant portions of the patient.

In the illustrated embodiment, the top surface 20 of the base 10 comprises a receiving portion 22. As illustrated, the receiving portion 22 is substantially flat. Generally, this receiving portion is shaped to match a bottom surface 37 of the bed 30, discussed further below. As discussed in regard to matching the lower surface of the base 10 to the patient, the upper surface 20 of the base 10 can be generally shaped to match the lower surface 37 of the bed 30. Additionally, as discussed above, the surfaces 20, 37 need not match precisely and may further be made flexible to accommodate differences. Generally, the top and bottom surfaces 20, 21 of the base 10 will substantially match each other in shape and size. However, in some embodiments it will be desirable for the top and bottom surfaces 20, 21 to differ, such as forming a shape generally similar to a truncated pyramid, providing additional surface area for adhesion to the anchor pad 6.

The receiving portion 22, as illustrated, further comprises tabs 13. The tabs 13 generally attach to the receiving portion 22 near the center of the base 10 and extend in the lateral direction. As will be discussed further below, the tabs 13 can be positioned and oriented generally collinear with the cleats 16. The illustrated tabs 13 terminate in semi-circular sections.

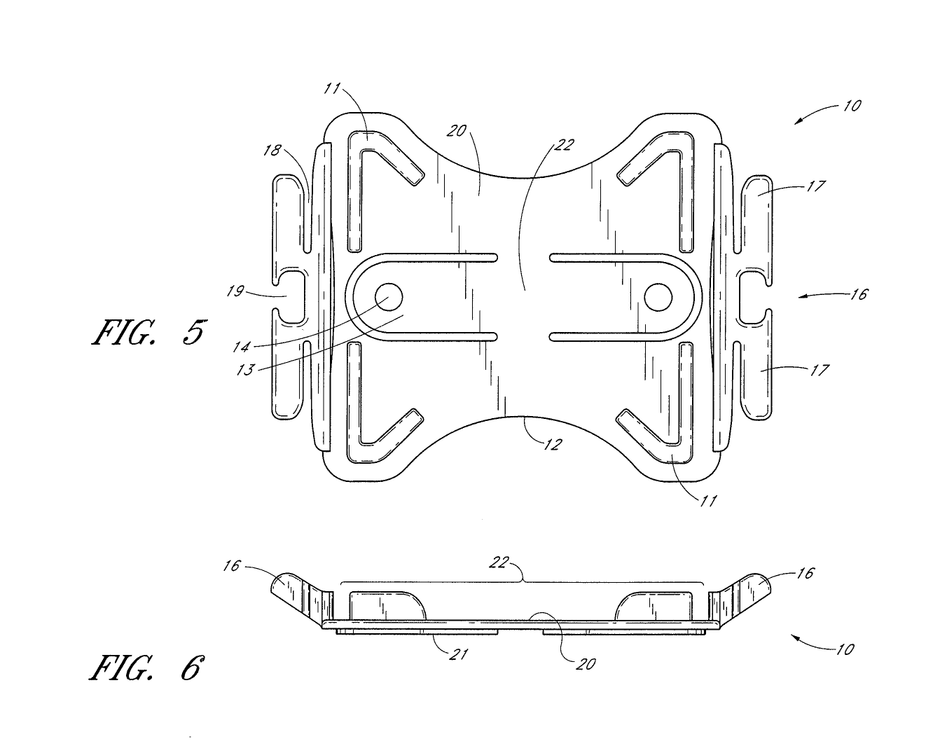

As best shown in FIG. 5, in the illustrated embodiment the tabs 13 terminate near the end of the receiving portion 22 and originate near the center of the receiving portion. Thus, the tabs 13 can span a length substantially equal to the lateral length of the receiving portion 22. As will be discussed further below, the elongated tabs 13 can advantageously bend at an angle when pressure is applied. Notably, the tabs 13 in the illustrated embodiment leave sufficient uncut material in the center of the receiving portion 22 so as not to cause the material to tear during normal use of the anchoring system 1 described herein. When a tougher material is used, the cuts defining the tabs 13 may come closer to the center of the base 10.

As illustrated, the tabs 13 further comprise holes 14. As will be discussed further below, a strand 25 can pass through the holes 14. When said strand 25 is tightened and is passing through one or more holes 14, it exerts a force on one or more tabs 13 at the hole(s) 14. Placement of the holes 14 near the distal end of the tabs 13 causes a greater torque to be applied to the tabs 13 as evaluated at the hole and relative to the axis of rotation at the base of the tabs, and thus causes a greater bend in the tabs. When less bending is desired, the tabs 13 can be shorter or the holes 14 can be placed further from the end of the tabs. Additional bending of the tabs 13 can cause the tabs to wrap about the constrained medical article 50 and thus apply an additional lateral retaining force.

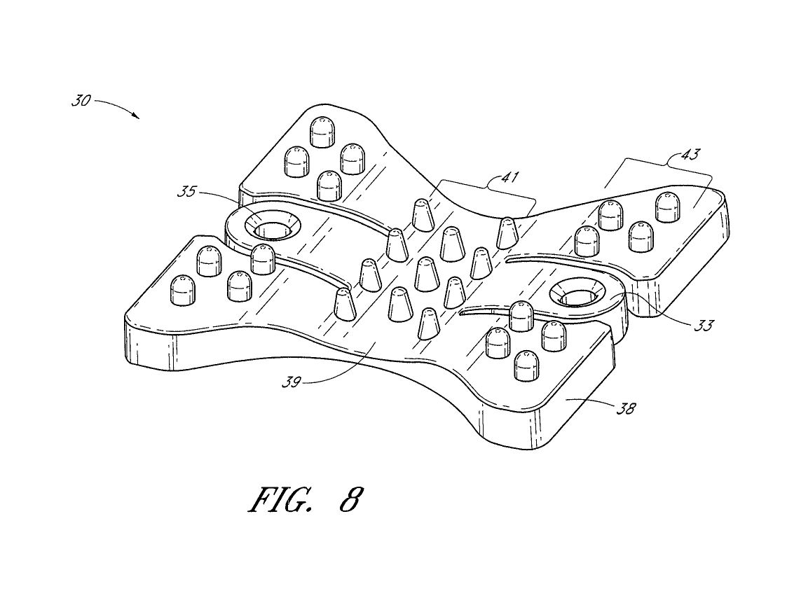

The holes 14, as illustrated in FIG. 7, further comprise radiused rims 15. As with other edges of the anchoring system 1, the radiused rims provide a smooth surface and remove potential sharp edges that may damage the strand 25. In this embodiment, the holes 14 on the tabs 13 on the base 10 are radiused only on the lower surface 20.

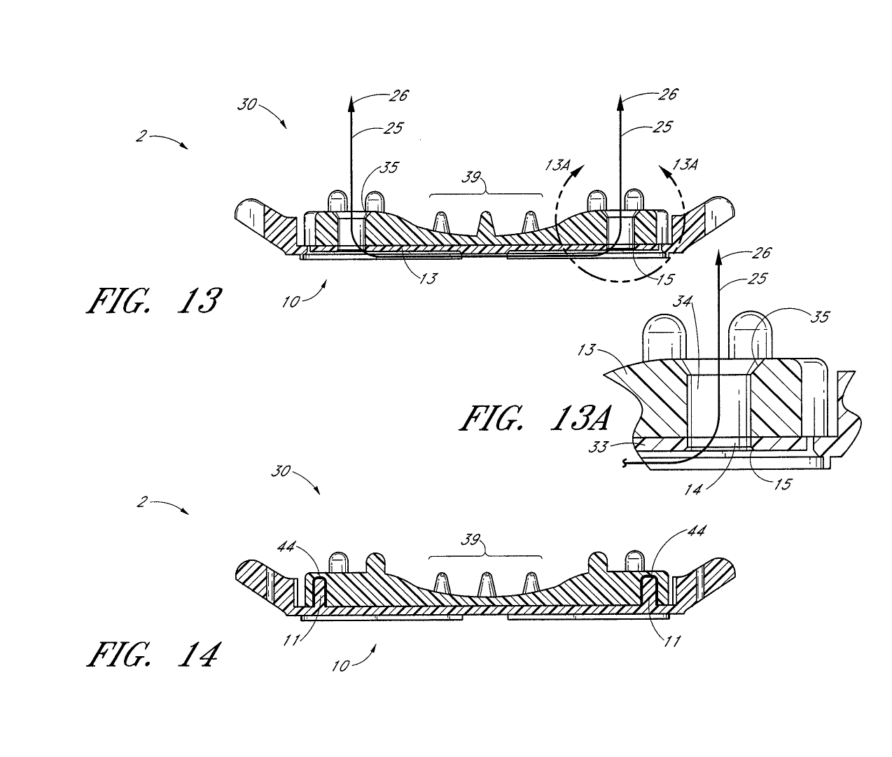

As shown in FIGS. 12-14 and discussed herein, in operation the bed 30 rests on top of the base, with similar tabs 33 and holes 34 aligned with the tabs 13 and holes 14 of the base 10. Thus, the aligned holes 14, 34 together comprise a single hole with radiused rims 15, 35 at the ends, but not at their seam. When the tabs 13 on the base 10 are thinner than the surrounding base (as discussed below) the radiused rims 15 of the base can be smaller than the radiused rims 35 of the bed 30. It will be clear from the disclosure herein that the holes need not be radiused in some embodiments, depending on the intended use, strength of abutting materials (e.g. the strand 25), and other factors.

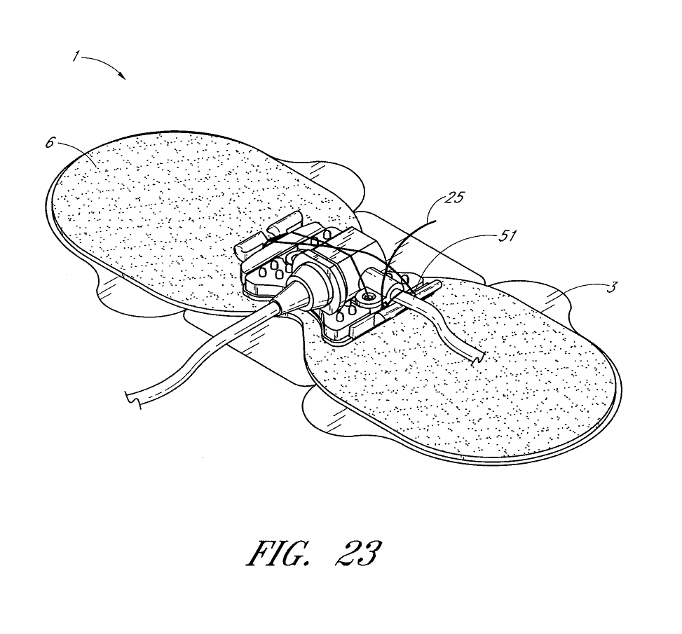

The strands 25 can also engage cleats 16 on the base 10. As illustrated, the cleats 16 are positioned on the lateral edges of the base 10. Each cleat 16 can comprise two ears 17, formed by longitudinal notches 18 and middle notches 19. The longitudinal notches 18 can be tapered to allow a strand 25 to self-lock when wrapped around the ears 17. Each ear 17 is sufficiently rounded and elongated to hold a strand 25 wrapped about said ear without slipping off or tearing. Further, as best shown in FIG. 5 the middle notch 19 is substantially rounded with a reduced opening. This shape of the middle notch 19 allows a strand 25 to be further held within the middle notch. In some embodiments, the middle notch 19 can also accept a portion of the medical article 50, such as a second branch 51, as depicted in FIGS. 22, 23. As shown in FIG. 23, the strand 25 can then wrap around the second branch 51 to further restrain the medical article 50. Further, as will be discussed below, the strand 25 can easily be wrapped about one ear, or around both ears of the cleats 16, as well as around any portion of the medical article 50.

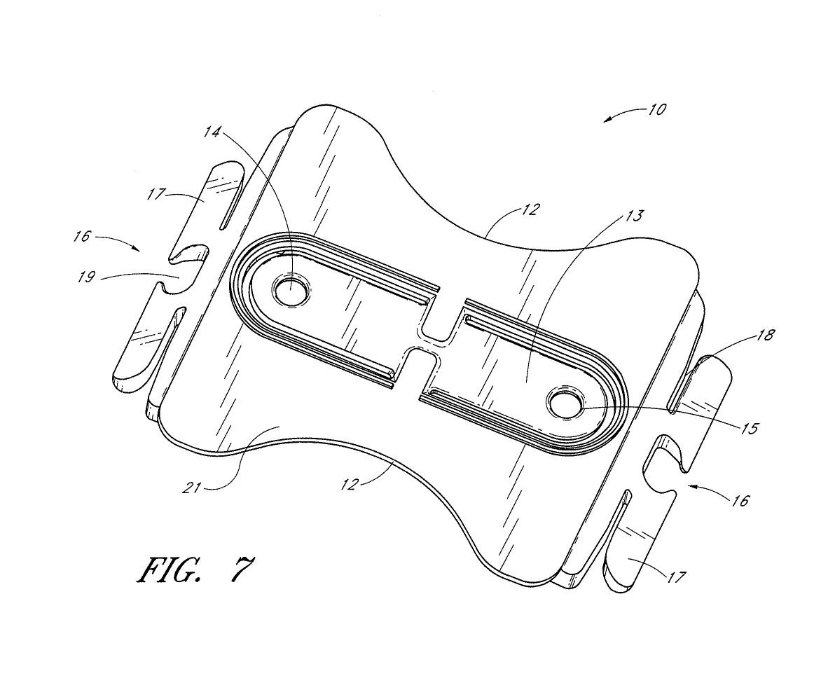

The cleats 16 in the illustrated embodiment generally project longitudinally, but also angle slightly upward in the transverse direction, as best seen in FIGS. 5, 6. In a preferred embodiment, the cleats 16 angle upward sufficiently to allow a finger beneath said cleats in the process of wrapping a strand 25. Although this clearance is desirable, it is not necessary. Alternatively, a cleat 16 with more than 2 ears may be provided, forming a star-like shape with many ears. It will be clear from the disclosure herein that other variations of the cleat can be provided, about which a strand 25 can be wrapped.

The surface and material of the cleats 16 can facilitate secure wrapping of the strand 25. The cleats 16 can comprise a smooth surface, reducing the possibility of tearing the strand 25 when wrapped about the cleat 16 under tension. To preserve tension in the strand 25, the cleat 16 can comprise an elastic material (as discussed with the tabs 13, 33 herein). To this effect, the material of the cleats 16 can differ from the material of the rest of the base 10 in some embodiments, for example to provide the cleats (and/or the tabs 13) with a more flexible material than the rest of the base.

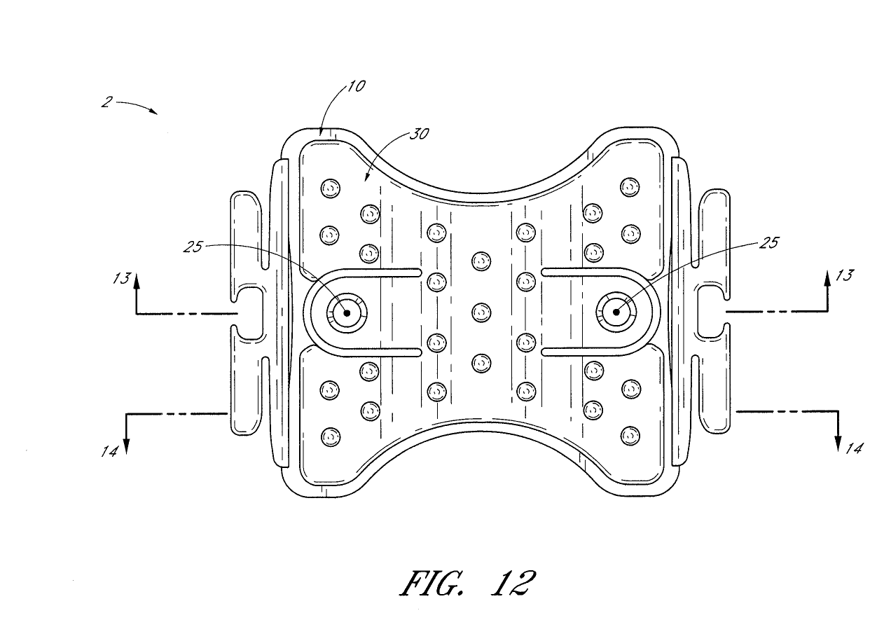

In the illustrated embodiment, the base 10 further comprises four keepers 11. The keepers 11 generally restrain the bed 30 when assembled to the base 10. The keepers 11 form raised walls along the peripheral corners of the receiving portion 22. The walls do not lie in a single plane, preventing sliding of the bed 30 when in contact with the base 10. Further, as shown in FIG. 4 the keepers 11 have a slight taper. The overall shape of the keepers 11 generally match recesses 44 in the bottom surface 37 of the bed 30, as shown in FIG. 11. In some embodiments, the keepers 11 can be slightly larger than the recesses 44, allowing for a press-fit when the anchoring system 1 is assembled. Of course embodiments of the anchoring system 1 need not include any keepers 11 and still fall within the scope of the invention.

It will be clear from the disclosure herein that alternative mechanisms for securing the base 10 and the bed 30 are possible. For example, the base 10 and bed 30 can be attached by adhesive, Velcro®, string, latch, lock, or some other reversible or irreversible connecting device. In the illustrated embodiment, the keepers 11 and the recesses 44 restrain the lateral and longitudinal motion of the bed 30 relative to the base 10. The illustrated keepers 11 restrain transverse movement by the friction of the press-fit. Further, in some embodiments the bed 30 can comprise sidewalls 38 that create a further press fit between the keepers 11 and cleats 16 of the base 10. The cleats 16 and keepers 11 may be positioned sufficiently close, or the side walls 38 of the bed may be sufficiently thick to create this additional frictional grip. Additionally, in many embodiments the strand 25 further holds the bed 30 to the base 10. For example, if the strand 25 moves through the holes 14, 34 in the bed 30 and the base 10, and then attaches to a cleat 16, then the bed and base will be attached. When the bed 30 must be restrained more or less reliably or stably in any given direction, a different or additional method of retention can be used.

As illustrated in FIGS. 8, 10, 13, 14, the bed 30 can comprise a groove 39 running through a central longitudinal axis of the bed 30, but need not. The groove 39 provides lateral stability to the secured medical article 50. The depth of the groove can be chosen to loosely match the shape of intended medical articles 50. For example, if a relatively tall medical article 50 is to be restrained by the anchoring system 1, the groove 39 can be made relatively deep. Nevertheless, it should be clear from the disclosure herein that most embodiments of the invention can accommodate a wide variety of sizes such as, for example, a relatively tall medical article 50 even when a channel 39 of a given embodiment is not designed specifically to accommodate a tall medical article.

To better accept a broad range of medical articles 50 in the retainer 9, the bed 30 can comprise a plurality of protrusions 40, 42. As illustrated, the protrusions can come in three sets: one set 41 of generally conical protrusions 40 and two sets 43 of tubular protrusions 42. The conical protrusions 40 can comprise a generally conical shape with a radiused tip, removing sharp edges. The tubular protrusions 42 can comprise a generally tubular shape, also with a radiused tip. The conical protrusions 40 can more easily extend into or surround a medical article 50 placed directly against said protrusions. The tubular protrusions 42 are generally stiffer due to their generally larger cross-section, and thus can better restrain a medical article 50 in side contact with said protrusions. Accordingly, in the embodiment illustrated in FIG. 1, the set of conical protrusions 41 resides generally in the groove 39 of the bed 30 and the sets of tubular protrusions 43 reside generally to the side of the groove 39 of the bed 30.

Other protrusions can be used in accordance with the invention. For example, another embodiment may comprise only one type of protrusions, such as conical protrusions 40. In another embodiment, the protrusions may be angled toward the center (as opposed to standing upright as in the illustrated embodiment). In another embodiment, the protrusions can comprise a larger or smaller radius, so as to better accommodate a medical article 50 with larger or smaller edges, holes, or other features. The protrusions can be formed integrally with the bed 30 or separately and press fit into holes formed in the bed 30. The protrusions can further comprise different shapes, such as generally resembling a pyramid, truncated pyramid, obelisk, square pylon, and other shapes. It will be clear from the disclosure herein that the protrusions may comprise other orientations, positions, sizes, and numbers in accordance with the invention herein.

As illustrated, the bed 30 further comprises corner wings 31 that extend longitudinally further than the central groove 39. The corner wings 31 can thus provide a broader base of support for the medical article 50, inhibiting rotation of the medical article 50 in the lateral-longitudinal plane using the central groove 39 as a pivot point.

The bed 30 can further comprise concave sections 32. The concave sections 32 leave a sufficient gap for a medical attendant or other person to easily access the medical article 50 while restrained by the anchoring system 1 without requiring removal of the medical article. Further, the concave sections 32 reduce the total area of the securement device 2, allowing placement in smaller areas. It will be clear from the disclosure herein that other sizes and shapes may be used to accomplish these goals in accordance with the invention. Further, it will also be clear from the disclosure that in some instances concave sections will not be needed, such as when access to the medical device 50 is not necessary while retained.

Like the base 10, the bed 30 comprises tabs 33 with holes 34 in the preferred embodiment. These holes 34 preferably comprise radiused rims 35. The base tabs 13 and holes 14 align with the bed tabs 33 and holes 34, allowing a flexible strand 25 to pass through said holes. The strand 25 can then wrap around a medical article 50 to retain said article in the retainer 9. For example, the strand 25 may pass through one pair of holes, circle about the medical article one complete revolution, and then wrap about a cleat 16 opposite said holes. Notably, the ability to wrap the strand 25 about the cleat 16 substantially removes necessity to tie a knot with the strand 25.

If the strand 25 is tightly wound, the strand can desirably be put in tension while retaining the medical article 50. When in tension, the strand 25 can retain the medical article 50 not only in the transverse and lateral dimensions, but also in the longitudinal dimension by frictional forces. Further, the illustrated embodiment comprises mechanisms to retain the strand 25 in tension. When the strand 25 passes through the holes 14, 34 it exerts a force on said holes and their respective tabs 13, 33, causing said tabs to deflect upwards. The deflected tabs 13, 33 can continue to deflect until the deflection causes a sufficient force to counteract the tension in the strand 25. Similarly, the cleat 16 can deflect inward under tension from the strand 25. With this deflection, the strand 25 may loosen yet still maintain tension as the tabs 13, 33 and/or cleat 16 relax only partially and continue to exert a counteracting force on the strand.

The deflection of the tabs 13, 33 and the cleats 16 can vary according to the tension in the strand 25, the material of the tabs and cleats, the distance from the point of application of the force to the pivot point, the angle of the force relative to the pivot point, the thickness of the tabs and cleats, and other factors. As an example, the tabs 13 of the base 10 have been thinned, as best shown in FIG. 13, causing them to bend more easily. In some embodiments the cleats 16 and/or tabs 13, 33 can be made thinner or thicker so as to increase or decrease deflection. Further, to the extent that they are intended to deflect, the cleats 16 and tabs 13, 33 should comprise an elastic material. The elasticity is desirably substantial enough to keep the strand 25 in tension if the strand 25 stretches or the medical article 50 moves while retained. However, the elasticity is desirably small enough to allow a sufficient reactionary force against the strand 25 to adequately retain the medical article 50.

Other tying arrangements achieve similar results. For example, the strand 25 can potentially go through only one pair of holes 14, 34, wrap about only one cleat 16, go through both holes, wrap about both cleats, or any combination of cleats and/or holes. The strand 25 may further pass through or wrap around a pair of holes 14, 34 or cleat 16 multiple times. Depending on the yield strength, elasticity, and other properties of the strand 25, as well as the desired restraining forces on the medical article 50, it may be desirable to provide more or less attachment points. For example, if a large restraining force is desired and a strand 25 with low yield strength is used, it will generally be desirable to use a larger number of attachment sites, distributing the load over a larger number of interfacing strands. As another example, if a lesser restraining force is needed, the strand 25 can attach to, e.g., a pair of holes 14, 34 and cleat 16 while only passing over a medical article 50 and not wrapping around it.

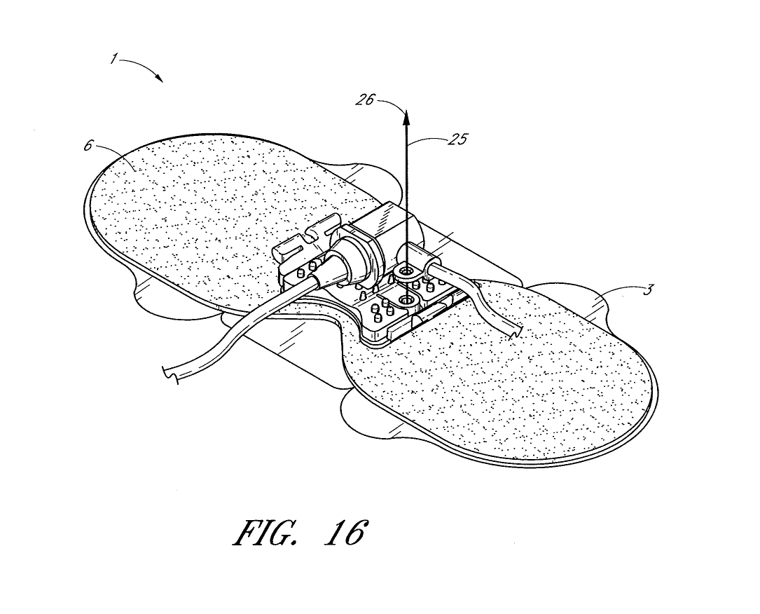

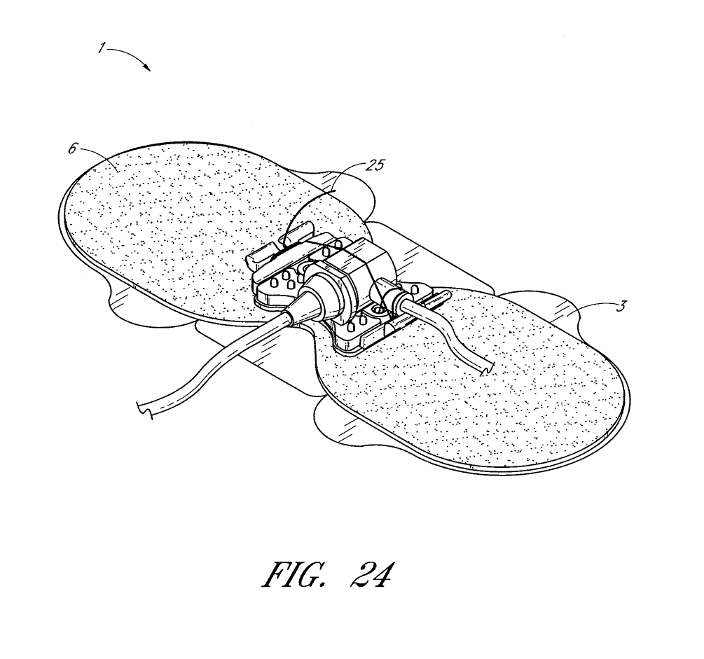

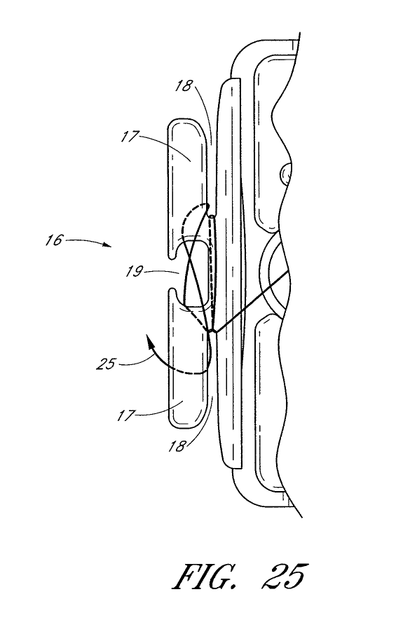

FIGS. 16, 22-24 depict multiple embodiment tying arrangements. As shown in FIG. 16, the strand 25 can initially pass through the holes 14, 34 in the base 10 and bed 30. As shown in FIG. 22, the strand 25 can then wrap over the medical article 50 and around a cleat 16 on the opposite side. For some purposes, this will provide sufficient securement of the medical article 50. However, in a further embodiment, the strand 25 can additionally wrap back over the medical article 50 and wrap about the other cleat 16, optionally also wrapping around the second branch 52 of the medical article 50 (optionally passing through the middle notch 19 of the cleat 16). In a similar tying arrangement depicted in FIG. 24, the strand 25 can first wrap about the second branch 52 and about the nearer cleat 16, and then go over the medical article 50 to wrap about the opposite cleat. Further tying arrangements are also possible and in accordance with other embodiments.

Additionally, some embodiments of the retainer 9 can comprise more cleats 16, tabs 13, 33, and holes 14, 34. Further tying arrangements with one or more strands 25 may be employed on embodiments with varying numbers of cleats 16, tabs 13, 33, and holes 14, 34. In the illustrated embodiment the cleats 16 and holes 14, 34 are substantially collinear, but in other embodiments they may be provided, for example, at the outer corners of the base 10 and bed 30. Providing holes 14, 34 and cleats 16 at the corners allow tying arrangements in “X” and or square patterns, as well as others. It will be clear from the disclosure herein that further tying arrangements may be incorporated depending upon the number and position of cleats 16, holes 14, 34, and analogous structures.

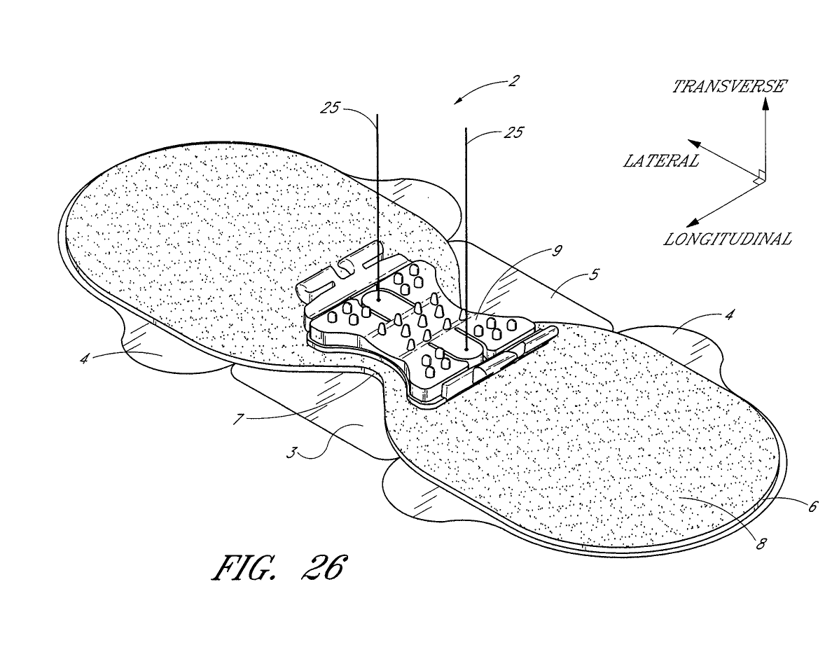

Like the variety of tying arrangements, a strand 25 can also wrap around a cleat 16 in a variety of ways. For example, if the strand 25 terminates at the cleat 16 the strand may be wrapped repetitively about the cleat 16 until the length of the strand is exhausted. To exhaust the length of the strand 25, in one embodiment the strand can first wrap around the base of the cleat 16, first extending across the inner side of said base before wrapping. The strand 25 can then tightly wrap around the base 16 a sufficient number of times to absorb a maximum tension expected to be applied to the strand 25 under normal operation of the medical article 50 and securement device 2. The remaining length of strand 25 can then tightly wrap around the cleat 16 in a repeating figure-eight wrapping path alternating between the ears 17 of the cleat 16 shown in FIG. 25. Before tightening the final figure-eight loop, the terminating end of the strand 25 can pass under the final figure-eight loop such that the subsequent tightening of the figure-eight loop presses the terminating end against the preceding loops and/or the cleat 16, preventing the strand 25 from loosening.

Further, the length of the strand 25 can be adjusted to reduce the number of figure-eight loops required. For example, the a medical attendant can cut the strand 25 after a single figure-eight loop and tightening of the terminating end to remove excess strand. Alternatively, the strand 25 can initially comprise a length suitable for a predetermined range of sizes of a medical article(s) 50.

The strand 25 may further comprise other properties. For example, the strand 25 can be relatively elastic or inelastic, potentially depending upon the elasticity of other elements such as the tabs 13, 33 and/or the cleat 16 for example. The strand 25 may further comprise a thickness sufficiently large so as not to kink a medical article 50 and allowing a medical attendant to easily grab the strand so as to unwrap it from the cleat 16. In some embodiments, said thickness can be as large as 5 mm. The strand 25 can have various diameter sizes depending upon the required strength of the strand 25.

The securement device 2 illustrated in FIG. 15 is the same as the device illustrated in FIG. 1 except that only one of the two ends of the strand 25 from FIG. 1 is shown in FIG. 15. The unused second end may be removed during manufacturing, removed at the point of application to the patient, or simply wrapped around the cleat 16. As shown in FIG. 15, the medical article 50 may comprise a tab 54 and hole 55. The tab 54 and hole 55 can be positioned to align with the holes 14, 34 and tabs 13, 33 of the retainer 9 (or alternatively, the holes and tabs on the retainer can be positioned to align with the hole on the medical article).

In the illustrated configuration in FIG. 15, the single end of the strand 25 secures the medical article 50 against movement with minimal wrapping. For example, referring to FIG. 15, 16, 22, a strand passing through the holes 14, 34, 55, over the medical article 50, and around an opposite cleat 16 may constrain the medical article 50. It will be clear from the disclosure herein that other tying arrangements can be used.

A second end of a continuous strand 25 or of a second strand 25 need not be used to secure the medical article 50. Of course a second end of the same strand 25 or of a second strand 25 could also be used to further secure the medical article 50.

The tab 54 can further serve as a gripping surface for a medical attendant or other person wishing to place, reposition, or remove the medical article 50. In some embodiments the tab 54 can extend sufficiently beyond the hole 55 such that the tab 54 can be gripped without occluding the hole 55.

In the illustrated embodiment, the strand 25 comprises a generally elongate form. The strand 25 may comprise materials such as string, rubber, plastic, silk, and the like. The strand may further comprise a single solid strand, a bundle of smaller strand 25, a single tube, a bundle of tubes, a generally flat strap, or similar structures or combinations thereof. As discussed herein, in most situations it will be desirable to keep the strand 25 in tension. Thus, an elastic material may be preferred for the strand 25.

In other embodiment, an inelastic material is employed. For example, the filament or strand may comprise No. 1 braided silk sutures. In such an embodiment, the material may be substantially non-extendible about its long axis. The use of such a material inhibits the strand 25 secured about the medical device 50 from loosening once secured to the cleat 16 or otherwise secured in place.

The strand 25 may further comprise materials or features that facilitate securement of the strand to the retainer 9. In the illustrated embodiment, the strand can easily wrap around the cleats 16 and through the holes 14, 34. However, in other embodiments alternative or additional attachment methods may be used such as Velcro sections on the strands 25, adhesive portions, hooks and/or loops, latches, and others.

The strands 25 may further comprise a hardened tip 26, as shown in FIG. 16. The hardened tips can comprise a generally pointed or conical shape so as to facilitate passing the strand 25 through the holes 13, 33. The hardened tip generally comprises a radiused top to remove unnecessarily sharp edges that may inadvertently damage the patient, the medical attendant, the anchoring system 1, the medical article 50, or another element or entity. In some embodiments, the strand 25 may comprise only one hardened tip, for example, if the other end of the strand 25 is permanently attached to a given portion of the securement device 2.

As illustrated, the medical article 50 can further comprise a junction 53 connecting two branches 51, 52. Embodiments of the invention without a cover (as illustrated) can accept medical articles 50 of nearly any size and shape. Therefore, the invention should not be construed as limited to the illustrated, exemplary embodiment of the medical article 50. As will be clear from the disclosure herein, the anchoring system 1 described is capable of securing a much broader range of items, and can further secure said items to people, animals, plants, and inanimate objects.

Although the illustrated embodiment depicts a single bed 30 to correspond with a single base 10, a system of retainers 9 can be provided in accordance with the invention. For example, a set of beds 10 with varying properties may be configured to fit on a single base 10. A medical attendant may thus first attach the anchor pad 6 and base 10, and then according to the attendant's immediate observations, select a bed 30 best suited for the situation. Similarly, if the situation changes, e.g. a different medical device 50 should be used, the attendant may not only change the medical device 50 but also the bed 30. Thus, the bed 30 can be specifically selected to fit another medical device 50 without requiring replacement of the anchor pad 6 and the base 10. Sets of beds may comprise varying sizes, shapes, protrusions, numbers of protrusions, numbers of tabs, numbers of cleats, materials, elasticities of tabs, and other relevant aspects.

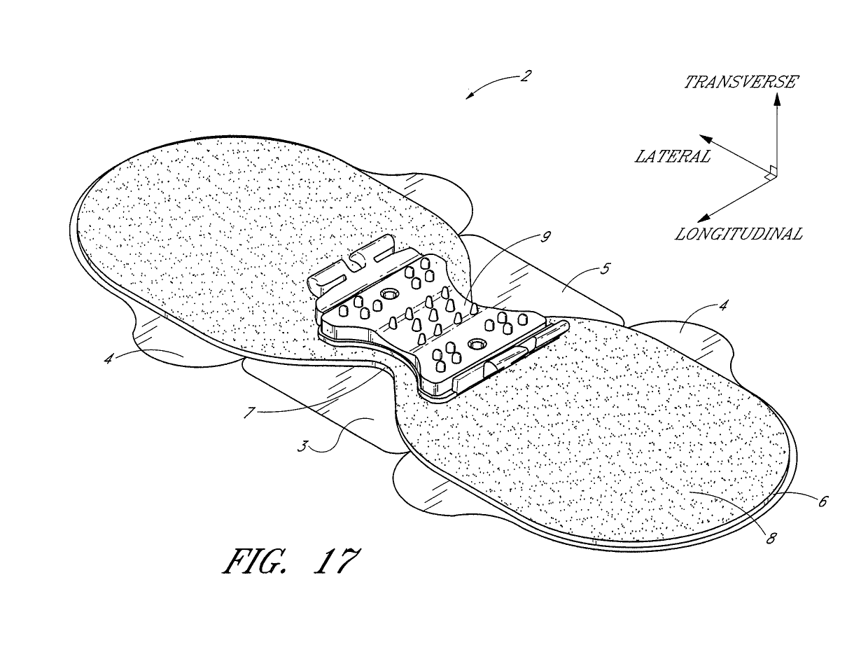

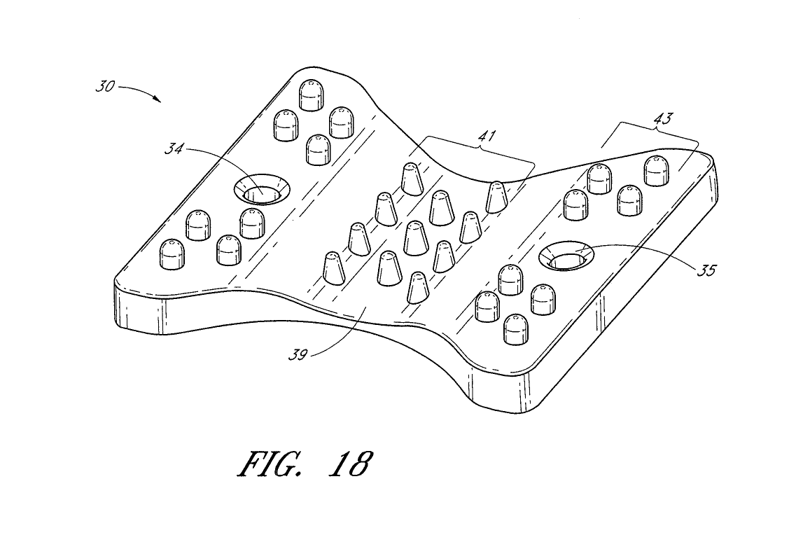

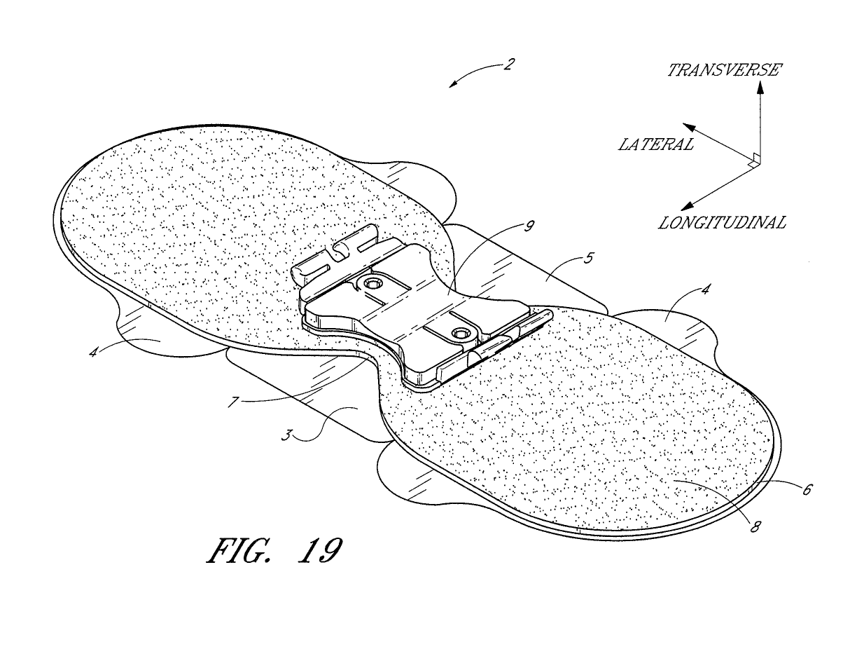

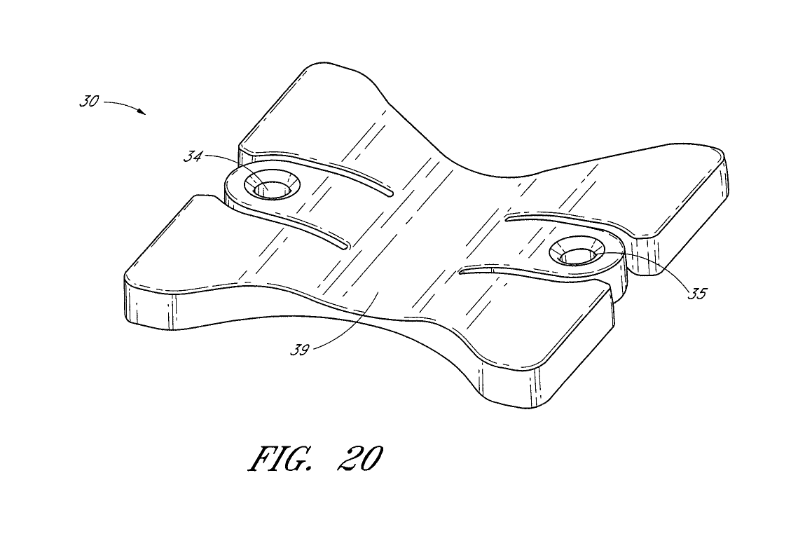

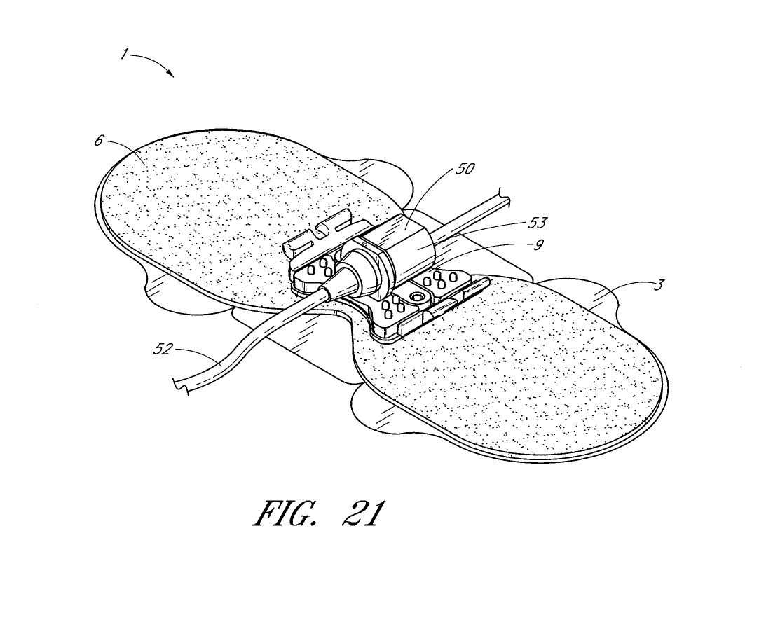

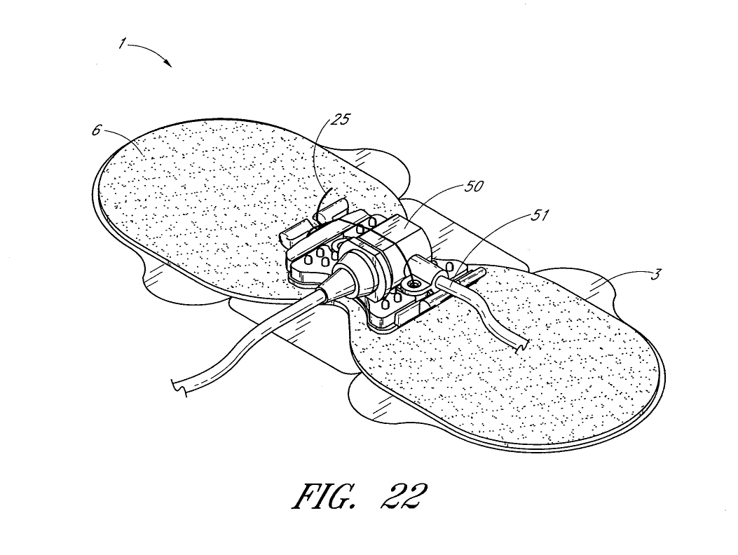

Although the foregoing systems and methods have been described in terms of certain preferred embodiments, other embodiments will be apparent to those of ordinary skill in the art from the disclosure herein. For example, FIGS. 17, 18 depict a securement device 2(a) without tabs 13, 33. In this embodiment a medical article 50 can still be retained by a strand 25 passing through the holes 14, 34. Tension in the strand can be maintained by means of, for example, flexibility in the cleats 16. Another example, FIGS. 19, 20 depict a securement device 2(b) without protrusions 40, 42. The securement device 2(b) can still secure a medical article 50 by means of the strand 25 engaging with other elements of the securement device. As another example, FIG. 21 illustrates an embodiment of the securement device 2 with a different medical article 50(a), prior to attachment with a strand 25.

In another preferred embodiment depicted in FIG. 26, multiple components of the securement device 2(c) may be formed as a unitary structure. For example, the strand 25 may be formed as a unitary structure with the retainer 9 (which in this embodiment also comprises a unitary structure), such that a strand portion extends from one or more locations on the securement (two locations depicted in FIG. 26). In such embodiments, the strands 25 can extend from the tabs 13 without the holes 14. Similarly, the retainer 9 can comprise a unitary structure without multiple layers and not include a strand 25 disposed between said multiple layers. An adhesive may be applied to the underside of such a unitary structure. In addition, when two or more strands 25 are used they can be tied together and thus the cleats can be easily removed while substantially preserving functionality.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. Thus, it is intended that the scope of the present invention herein disclosed should not be limited by the particular disclosed embodiments described above, but should be determined only by a fair reading of the disclosure and the claims that follow.

What is claimed is:

1. A securement device for a medical article comprising:
an anchor pad having a bottom surface and a top surface, at least a portion of said bottom surface being covered by adhesive;
a base and a bed, the base being supported by said anchor pad;
at least one biasing member, at least a portion of said biasing member being disposed below said bed and having a generally fixed base and a distal portion that flexes relative to said base;
at least one cleat; and
at least one filament coupled to said biasing member and having a free end, said free end being configured to be tied about a portion of said medical article and secured relative to said cleat.

2. The device of claim 1, wherein said base comprises polycarbonate material.

3. The device of claim 1, wherein said base comprises a plastic material.

4. The device of claim 1, wherein said bed comprises a polymeric material.

5. The device of claim 1, wherein said anchor pad comprises a fabric material overlaid by a hydrocolloid adhesive material.

6. The device of claim 1, wherein said filament comprises a flexible material.

7. The device of claim 1, wherein each of said base and said bed has at least one hole extending therethrough, said filament extending through said holes.

8. The device of claim 7, wherein said hole in said base is at least partially aligned with said hole in said bed.

9. The device of claim 8, wherein said hole in said bed is configured to align with a hole in a medical device.

10. The device of claim 1 further comprising at least two cleats positioned generally opposite one another.

11. The device of claim 1, wherein said cleat is formed integrally with the retainer.

12. The device of claim 1, wherein said cleat is attached to the anchor pad.

13. The device of claim 1, wherein said base is harder than said bed.

14. The device of claim 1, wherein the cleat comprise at least one ear.

15. The device of claim 14, wherein said ear is formed by at least one longitudinal notch and a middle notch.

16. The device of claim 15, wherein the longitudinal notch is tapered.

17. The device of claim 14, wherein said ear has a rounded and elongated shape.

18. A retainer for securing a medical article comprising:
a support having an upper member supported at least in part by a lower member, said lower member being harder than said upper member;
at least one aperture;
a cleat extending from said lower member and having at least one slot; and
a filament coupled to said lower member through said at least one aperture and having a free end, at least a portion of said filament being configured to wrap about said cleat and engage with said slot.

19. The retainer of claim 18, wherein said cleat comprises an elastic material.

20. The retainer of claim 18, wherein said cleat generally extends in a longitudinal direction while angling upward in a transverse direction.

21. A securement device for a medical article comprising:
a retainer comprising at least two materials, at least one of the two materials being softer than the other one of the materials;
at least one cleat having a slot; and
at least one filament permanently coupled to said retainer and having a free end, said free end being configured to be wrapped about a portion of said medical article and secured through said slot of said cleat.

22. A method for securing a medical article comprising:
providing a securement device having a strand, at least one cleat, and an adhesive bottom surface, the adhesive bottom surface being covered by at least three individual release liners;
removing a first release liner to uncover a central portion of the adhesive bottom surface;
adhering the central portion to the skin of a patient;
wrapping the strand about at least a portion of a medical article;
securing the strand to the at least one cleat;
removing a second release liner so as to expose a first outer portion of the adhesive bottom surface;
adhering the first outer portion to the skin of the patient;
removing a third release liner so as to expose a second outer portion of the adhesive bottom surface; and
adhering the second outer portion to the skin of the patient.
